(19) United States
(12) Patent Application Publication
(10) Pub. No.: US 2003/0216696 A1
(43) Pub. Date: Nov. 20, 2003

(54) Setting element for an injection device

(76) Inventor: Thomas Hegewald, Baiersdorf (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/438,226

(22) Filed: May 14, 2003

(30) Foreign Application Priority Data
May 15, 2002 (DE) 10221781.5

(51) Int. Cl. A61M 5/00

(52) U.S. Cl. 604/207

(57) ABSTRACT

The present invention relates to a setting element for an injection device, in which a dose of a liquid to be injected can be set in predetermined steps by moving a setting element a corresponding distance, the movement of the setting element follows a trajectory with predetermined locking points along a cam, and the trajectory can be traveled in two directions of movement, by virtue of the fact that the trajectory, at each point, has finite gradients in both directions.

The invention relates to a setting element for an injection device.

The Applicant markets an injection device under the name OptiPen® for insulin injection. This device has a setting element with which, by turning it, a specific dose of insulin can be set. A sawtooth-shaped trajectory is followed upon turning the setting element relative to a cam. When the (round) cam rests both on a perpendicular surface and also on an oblique surface of the sawtooth-shaped trajectory, a locking point is defined. When this locking point is reached, a click can be heard from the perpendicular surface. This proves advantageous particularly in injection devices for insulin injection, because diabetes is also often accompanied by impaired vision. Based on the number of “saw teeth” on the trajectory about which the setting element is turned, an insulin dose to be injected can be set in corresponding steps.

### SUMMARY OF THE INVENTION

The problem on which the present invention is based is that of improving the setting possibilities for the insulin dose.

According to the present invention, this object is achieved in that the trajectory can be traveled in two directions of movement, by virtue of the fact that, at each point, the trajectory has finite gradients in both directions of movement.

It proves advantageous if, when setting the amount of insulin to be injected, it is also possible to “reset” an insulin amount which has been accidentally set too large. In the known device, this is not possible because the perpendicular surfaces (infinite gradient) of the sawtooth-shaped trajectory do not allow the setting element to be turned in the direction of a smaller dose of insulin.

Since, according to the present invention, the sawtooth-shaped trajectory is changed so that finite gradients are present in both directions, it is advantageously possible also to reduce an amount of insulin which has been accidentally set too large.

Hitherto, in such an eventuality, an injection first had to be carried out without inserting the needle. The corresponding amount of insulin was then lost. Thereafter, a new setting could begin.

### BRIEF DESCRIPTION OF THE FIGURES

An illustrative embodiment of the invention is shown in the drawing. In this drawing:

FIG. 1 shows a cross section of the trajectory according to the invention, and

FIG. 2 shows a cross section of a trajectory according to the prior art.

### EMBODIMENTS OF THE INVENTION

An embodiment of the invention provides a setting element for an injection device, in which a dose of a liquid to be injected can be set in predetermined steps by moving a setting element a corresponding distance, the movement of the setting element following a trajectory (1, 2) with predetermined locking points along a cam, wherein the trajectory can be traveled in two directions of movement, by virtue of the fact that the trajectory, at each point, has finite gradients (1, 2) in both directions of movement.

An embodiment of the invention provides the said setting element for an injection device, wherein, starting from a locking point, the gradient (1) in the direction of a larger dose quantity differs from the gradient (2) in the direction of a smaller dose quantity.

An embodiment of the invention provides the said setting element for an injection device, wherein the gradient in the direction of a larger dose quantity, starting from a locking point, differs from the gradient in the direction of a smaller dose quantity.

It is thus possible for the user to monitor, by feel, whether the setting element is being turned in the direction of a larger amount of insulin or in the direction of a smaller amount of insulin. It proves advantageous, in this connection, if the gradient in the direction of a larger amount of insulin is less pronounced than the gradient in the direction of a smaller amount of insulin. This proves advantageous if setting the dose starting from a “0” amount of insulin.

An embodiment of the invention provides the said setting element for an injection device, wherein an acoustic signal can be emitted by the cam (3) when a locking point is reached.

An embodiment of the invention provides the said setting element for an injection device, wherein an acoustic signal can be emitted by the cam when a locking point is reached.

The advantage of this is that even users with impaired vision can detect the dose set simply by counting the acoustic signals.

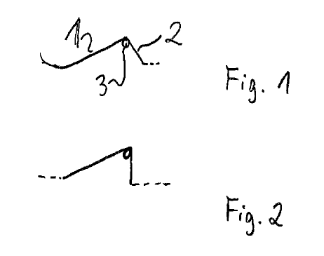

FIG. 1 shows a cross section of a trajectory which can for example be formed as a circumferential collar on an annular setting element. It can be seen from FIG. 1 that, in particular also at the locking point which is defined by the illustrated position of the cam 3, the trajectory has a finite gradient in both directions of rotation. This means that, in this position too, the setting element can be moved in both directions of rotation.

It will further be seen that the two gradients 1 and 2 are inclined to different extents. In this way, the user can advantageously feel whether he is turning the setting element in the direction of larger or smaller doses.

An acoustic signal is advantageously also emitted when the locking point is reached.

FIG. 2 shows a corresponding trajectory according to the prior art. At the locking point, the gradient is infinite in respect of one direction of rotation. This means that turning in this direction is not possible.

1. A setting element for an injection device, in which a dose of a liquid to be injected can be set in predetermined steps by moving a setting element a corresponding distance, the movement of the setting element following a trajectory (1, 2) with predetermined locking points along a cam, wherein the trajectory can be traveled in two directions of movement, by virtue of the fact that the trajectory, at each point, has finite gradients (1, 2) in both directions of movement.

2. The setting element as claimed in claim 1, wherein, starting from a locking point, the gradient (1) in the direction of a larger dose quantity differs from the gradient (2) in the direction of a smaller dose quantity.

3. The setting element as claimed in claim 1, wherein an acoustic signal can be emitted by the cam (3) when a locking point is reached.
